(19) United States
(12) Patent Application Publication
(10) Pub. No.: US 2003/0060672 A1
(43) Pub. Date: Mar. 27, 2003

(54) Production of hydrocarbons from mercaptans

(75) Inventor: Israel E. Wachs, Bridgewater, NJ (US)

Correspondence Address: BANNER & WITCOFF, 1001 G STREET N W, SUITE 1100, WASHINGTON, DC, 20001, US

(73) Assignee: LEHIGH UNIVERSITY, Bethlehem, PA

(21) Appl. No.: 10/170,232

(22) Filed: Jun. 13, 2002

Related U.S. Application Data
(60) Provisional application No. 60/298,076, filed on Jun. 15, 2001.

(51) Int. Cl. C07C 1/00

(52) U.S. Cl. 585/638

(57) ABSTRACT

A method wherein a gas stream containing a mercaptan is passed in contact with a SAPO catalyst comprising to convert at least a portion of the mercaptan to a hydrocarbon.

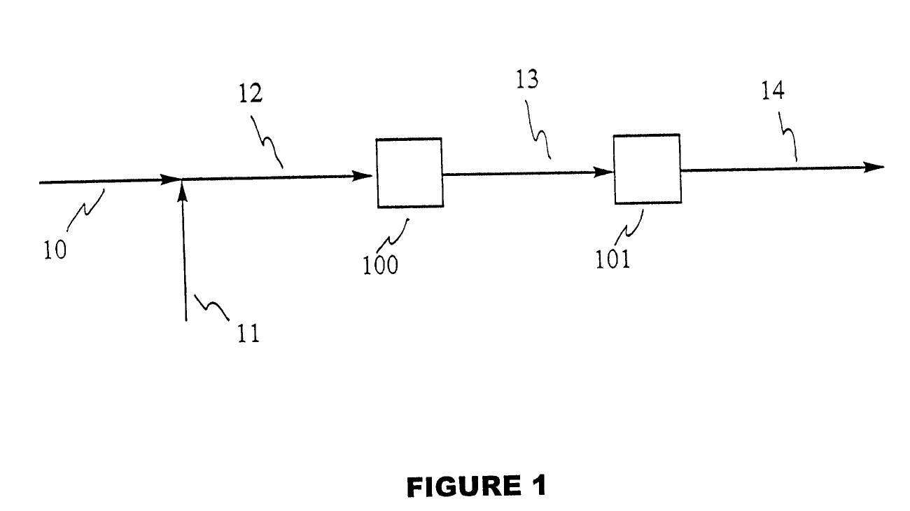

### BACKGROUND OF THE INVENTION

1. Field of the Invention

This invention broadly relates to a process for producing hydrocarbons from mecaptans, and especially for producing lower alkanes from methanethiol. More particularly, this invention provides a method wherein a gas stream containing a mercaptan, preferably methanethiol, is passed in contact with a catalyst comprising a silicoaluminophosphate (SAPO) molecular sieve, often in the presence of a diluent, for a time sufficient to convert at least a portion of the mercaptan to a hydrocarbon.

2. Description of Related Art

Mercaptans, such as methanethiol or methyl mercaptan (CH₃SH), dimethyl sulfide (CH₃SCH₃) and dimethyl disulfide (CH₃SSCH₃), are found as impurities or are produced as a generally undesirable by-product in a wide variety of industrial gas streams. These reduced sulfur compounds are exceedingly malodorous (detectable at parts per billion [ppb] levels), are extremely hazardous, and often are considered a pollutant. It is expected that their emission will be subject to more restrictive regulation in the future.

U.S. Pat. No. 4,677,243 describes a process for the production of light olefins, i.e., olefins having not more than four carbon atoms, from a feedstock comprising aliphatic hetero compounds, preferably methanol, ethanol, dimethyl ether, diethyl ether or mixtures thereof, in the presence of a silicoaluminophosphate (SAPO) molecular sieve catalyst, at a temperature of from about 200° C. (392° F.) to 700° C. (1292° F.).

Applicant now has made the discovery that certain molecular sieve catalysts, well-known as SAPO catalysts, can be used to convert mercaptans, and especially methanethiol, to hydrocarbons, including lower alkanes in a heterogenous catalytic process.

### BRIEF DESCRIPTION OF THE DRAWINGS

The sole figure is a highly simplified schematic drawing illustrating the process of the present invention. Those skilled in the art recognize that a variety of process steps and equipment, including heat exchangers, pumps, compressors, control systems, separation equipment and the like, which are not shown in the schematic representation, will be needed to actually implement the process of the present invention. Those skilled in the art fully understand how to so-adapt the process for actual use.

### BRIEF DESCRIPTION OF THE INVENTION

The present invention is directed to a method for converting a mercaptan feedstock, especially methanethiol (CH₃SH), supplied in a gaseous feed, to hydrocarbons, including especially lower alkanes. The process involves flowing a gaseous stream containing a mercaptan and optionally an inert diluent in contact with a catalyst comprising a SAPO molecular sieve for a time sufficient to convert at least a portion of the mercaptan to a hydrocarbon, and then recovering the hydrocarbon as a product separate from the constituents of the gas stream.

SAPO molecular sieve catalysts useful for practicing the present invention are well known in the prior art. The ability of these catalysts to convert mercaptans to hydrocarbons was unexpected

### DETAILED DESCRIPTION OF THE INVENTION

As noted, the present invention is directed to a method for converting mercaptans, especially methanethiol (CH₃SH), to hydrocarbons, particularly lower alkanes. The mercaptan is supplied in a gaseous feed optionally containing an inert diluent. In accordance with the present invention, the gas stream containing the mercaptan and the optional diluent contacts the SAPO catalyst at a temperature in the range of 200° to 700° C., usually in the range of 250° to 600° C. and most often in the range of 300° to 550° C. The mercaptan generally is contacted with the catalyst in the presence of an inert diluent such as helium, argon, nitrogen, carbon monoxide, carbon dioxide, hydrogen, water, paraffins and other hydrocarbons (such as methane and the like), aromatic compounds, or mixtures thereof. The diluent is present in the gaseous feed in an amount between about 1 and about 99 molar percent, based on the total number of moles of all feed and diluent components fed to the reaction zone (or catalyst). The amount of diluent added to the mercaptan feedstock will depend in part on the particular SAPO catalyst selected and the reaction temperature. Usually, the molar ratio of mercaptan feed-to-diluent (feed:diluent) is about 1:1 to about 1:4.

The process is conducted with the mercaptan in the vapor phase, such that the mercaptan feedstock is contacted in the vapor phase in a reaction zone with a silicoaluminophosphate (SAPO) molecular sieve at process conditions effective to produce the desired hydrocarbon products, i.e., at an effective temperature, pressure, and flow rate and, optionally, an effective amount of diluent. The contacting of the mercaptan with the SAPO catalyst at the appropriate conditions causes a conversion of the mercaptan to hydrocarbons, predominately lower alkanes and possibly lower olefins. Lower alkanes include methane, ethane and propane. Lower olefins include ethylene and propylene. The mercaptan reactant in the gaseous feed stream generally will comprise at least about 0.01 mole %, preferably at least 0.1 mole % and most often at least about 1 mole % of the gas feed stream, although much higher concentrations may be and often are employed.

As noted above, the gas feed stream contacts the SAPO catalyst at a temperature in the range of 200° to 700° C., usually in the range of 250° to 600° C. and most often in the range of 300° to 550° C. A maximum temperature of about 500° C. will often be suitable. Lower temperatures generally result in lower rates of reaction, and the formation of desired hydrocarbon products may become markedly slower. At much higher temperatures, however, the process may not form an optimum amount of desired hydrocarbon products.

A mercaptan for use as a feedstock in the present invention can be recovered as a by-product from other chemical processes or can be produced from available raw materials. Processes producing mercaptans as a byproduct are well known and include for example paper-making (pulping) via the Kraft process. Mercaptan for practicing this invention also can be prepared by contacting a and gas stream containing hydrogen sulfide and a carbon oxide, preferably carbon monoxide, with a catalyst comprising a supported metal oxide, such as an oxide of a metal selected from the group consisting of vanadium (V), niobium (Nb), molybdenum (Mo), chromium (Cr), rhenium (Re), tungsten (W), manganese (Mn), titanium (Ti), zirconium (Zr) and tantalum (Ta) and mixtures thereof, which converts the carbon oxide and hydrogen sulfide to mercaptans, (primarily methanethiol (CH₃SH) and a small amount of dimethyl sulfide (CH₃SCH₃)). Such catalysts preferably contain the metal oxide as a monolayer. Ratcliffe et al., U.S. Pat. No. 4,570,020, for example, describes a catalytic process for producing methanethiol (CH₃SH) from a gaseous feed comprising a mixture of carbon monoxide (CO) and hydrogen sulfide (H₂S). According to this patent, the gaseous mixture is contacted, at a temperature of at least about 225° C., with a catalyst comprising a metal oxide of a metal selected from the group consisting of vanadium (V), niobium (Nb), and tantalum (Ta) and mixtures thereof supported as an oxide layer on titania. The source of the mercaptan feedstock is not a critical aspect of the broadest features of the invention and any source of mercaptan should be suitable. However, a particular preferred embodiment of the present invention is directed to producing hydrocarbon from hydrogen sulfide and carbon oxide feedstocks.

The operating pressure for the catalytic reactor also is not critical, though operation at a greater than atmospheric pressure will generally be practiced. In its broadest aspects, the process can be carried out over a wide range of pressures including an autogeneous pressure. At pressures between about 0.001 atmospheres (0.76 torr) and about 1000 atmospheres (760,000 torr), hydrocarbon products will be formed although the optimum amount of a particularly desired product will not necessarily form at all pressures. A more typical operating pressure is between about 0.01 atmospheres (7.6 torr) and about 100 atmospheres (76,000 torr). The pressures referred to herein for the process are the mercaptan partial pressures and thus are exclusive of any inert diluent that may be present. Thus, these pressures refer to the partial pressure of the feedstock as it relates to the mercaptan constituent. Pressures outside the stated range are not excluded from the scope of this invention, although such pressures do not fall within the most common embodiments of the invention. At the lower and upper end of the pressure range, and beyond, the selectivities, conversions, and/or rates at which hydrocarbon products are produced may not be optimal.

The reaction is exothermic. As recognized by those skilled in the art a variety of reactor designs may be employed to accommodate the necessary mass and heat transfer processes for effective operation. In its broadest aspects, the catalytic process can be conducted as a batch, semi-continuous or continuous reaction. Fixed bed, moving bed and fluidized bed designs can optionally be employed. The reaction is conducted for a period of time sufficient to produce the desired hydrocarbon products. In general, the residence time employed to produce the desired product(s) can vary from seconds to a number of hours. It will be readily appreciated that the residence time will be determined to a significant extent by the reaction temperature, the particular SAPO molecular sieve selected, the mercaptan feedstock flow rate, mercaptan partial pressure and other process design characteristics selected. To achieve a higher selectivity in the conversion of mercaptan to hydrocarbons it is important to maintain the flow rate of mercaptan per unit mass of SAPO catalyst in the range of 1 to 1000, more usually 10 to 1000, and often 10 to 100 cubic centimeters (assessed under standard conditions of temperature and pressure (STP)) of mercaptan per gram of active SAPO catalyst per hour (excluding inert ceramic components or other inert catalyst support material). Generally, higher reaction temperatures permit higher flow rates.

As used herein, the term “selectively” is intended to embrace the conversion of at least 1% of the mercaptan, preferably at least 10% of the mercaptan, more usually at least 50% of the mercaptan and most preferably at least 70% of the mercaptan which contacts the SAPO catalyst to hydrocarbons. Selectivity, as that term is used herein, is determined by the percentage of hydrocarbons in the mercaptan conversion products as a proportion of the other carbon-containing mercaptan products.

The reaction mixture leaving the reactor is generally subject to further processing in a conventional manner. The hydrocarbon products can be recovered from the gaseous reaction products using anyone of a number of ways known to those skilled in the art. For example, the hydrocarbon/diluent gas mixture may be compressed and the hydrocarbon condensed by cooling at elevated pressures, or the hydrocarbon may be absorbed by a higher molecular weight alkane and the desired hydrocarbons stripped out by reheating the absorber product. Numerous ways for processing the hydrocarbon product will be apparent to those skilled in this art.

For obtaining higher yields and selectivities in the conversion of mercaptans to hydrocarbons, it may be desirable to conduct the reaction such that only a partial reaction takes place in a single pass through a reactor. For example, the pressure, temperature, composition of the starting gas mixture, the amount of SAPO catalyst and/or the rate of flow can be varied to cause a partial conversion of the mercaptan feed. The reactor effluent gas remaining after optional inter-stage separation of the hydrocarbons can then be delivered either by recycle or into a second reactor stage for additional reaction. It may be desirable to add to this gas an amount of mercaptan to replace all or some of the mercaptan that has been consumed. If the gas is recirculated, inert gases and other by-products, will concentrate in any recycled gas, and any excessive accumulation of these gases can be prevented by a continuous or discontinuous side-stream removal. It is also desirable to replace any removed exhaust gas with an equal amount of fresh gas to maintain operating pressures and flow rates.

The silicoaluminophosphate (SAPO) molecular sieve catalysts used in this invention are well-known and commercially available materials. Generally described as microporous crystalline silicoaluminophosphates, the pores of such materials are uniform and have nominal diameters of greater than about 3 Angstroms. The SAPO molecular sieves have a microporous crystal framework structure of PO₂⁺, AlO₂⁻ and SiO₂ tetrahedral units. The composition (anhydrous) is represented by the general formula:

mR: (SiₓAl_{y}P_{z})O₂

wherein “R” represents at least one organic templating agent present in the intracrystalline pore system, “m” represents the moles of “R” present per mole of (SiₓAl_{y}P_{z})O₂ and “m” has a value of from zero to 0.3, such as from 0.02 to 0.3, the maximum value in each case depending upon the molecular dimensions of the templating agent and the available void volume of the pore system of the particular SAPO species involved, as recognized by those skilled in the art, and “x,” “y,” and “z” represent the mole fractions of silicon, aluminum, and phosphorous, respectively. Representative SAPO molecular sieves, as described in U.S. Pat. No. 4,440,871 (and incorporated herein by reference), include SAPO-5, SAPO-11, SAPO-17, SAPO-20, SAPO-31, SAPO-34, SAPO-35, SAPO-37, SAPO-40, SAPO-41, and SAPO-42. SAPO-34, which has a pore size large enough to adsorb xenon, but small enough to exclude isobutene, is a suitable catalyst for use in the present invention. See also U.S. Pat. Nos. 4,677,243 and 5,248,647 for additional information about SAPO molecular sieves, which patents are incorporated herein by reference.

The SAPO catalyst can be supplied in many forms. In one embodiment, the silicoaluminophosphate molecular sieve is supplied in the form of particles formed with binder materials such as silica, alumina, silica-alumina, silica-magnesia, silico-zirconia, silica-thoria, silica-berylia, silica-titania, as well as ternary compositions, such as silica-alumina-thoria, silica-alumina-zirconia and with clays. The relative proportions of the above-noted binder materials and the silicoaluminophosphate may vary widely, with the silicoaluminophosphate content ranging between about 1 and about 99 percent by weight of any composite. In another embodiment, the SAPO catalyst can be supported on a monolithic support, i.e., on a low surface area support, or on a support comprising a material with little surface porosity. Such materials include, but are not necessarily limited to glass, metals, and enamel, which are substantially devoid of surface porosity. Monolithic supports also may be made of materials which have minute surface porosity but which are not impregnated in the usual sense by treatment, e.g., with silica sols. Such materials include, but are not necessarily limited to porcelain, fused alumina, fused silica, mullite, beryl, zirconia, dense sintered alumina, chromia, spinel, magnesia, fused magnesia, and titania. The support may be in the shape of a honeycomb, a sponge, pellets, granules, spheres, bars, rods, tubes, rolls, spirals, screens, beads, coils, or any of the conventional shapes in the art. Please refer to U.S. Pat. No. 5,925,800, incorporated herein by reference, for additional information concerning such support material.

Now with reference to the sole drawing, the process of the present invention will be briefly described. A mercaptan feedstock 10, at a pressure of about 1 to about 5 atmospheres, comprising at least about 1000 ppm methanethiol, is admixed as needed with a diluent 11. The admixture of methanethiol and diluent then is passed via line 12 through a heat exchanger 100 to preheat the feedstock/diluent admixture to a desired reaction temperature, i.e., most often to a reaction temperature ranging from about 300° C. to about 550° C., and provide a preheated feedstream 13. The preheated feedstream 13 is passed to a reaction zone 101 containing a SAPO catalyst selective for the conversion of at least a portion of the mercaptan in the reactor feedstream into lower alkanes and possibly lower olefins and to produce a reactor effluent stream 14. The reactor effluent stream 14, comprising the hydrocarbon product, such as methane, water, and other carbon-containing and sulfur-containing products, is treated by means not shown to recover the desired hydrocarbon products.

As noted above, a particularly preferred embodiment of the present invention involves a process for producing hydrocarbons, especially lower alkanes, from a gas stream containing a mixture of carbon oxide (CO and/or CO₂) and hydrogen sulfide (H₂S). As used throughout the specification and claims, the term “carbon oxide” is intended to embrace carbon monoxide (CO), carbon dioxide (CO₂) and mixtures thereof. A suitable gas stream will often contain a sizable fraction of both carbon monoxide (CO) and carbon dioxide (CO₂).

The gas stream containing carbon oxide and hydrogen sulfide (H₂S) is first passed in contact with a catalyst comprising a supported metal oxide of a metal selected from the group consisting of vanadium (V), niobium (Nb), molybdenum (Mo), chromium (Cr), rhenium (Re), tungsten (W), manganese (Mn), titanium (Ti), zirconium (Zr) and tantalum (Ta) and mixtures thereof to convert said carbon monoxide and hydrogen sulfide to mercaptans, (primarily methanethiol), and the mercaptans are then passed in contact with a SAPO catalyst as described above for a time sufficient to convert at least a portion of the mercaptans to hydrocarbons, such as lower alkanes.

This aspect of the invention involves flowing the gaseous stream containing carbon oxide and hydrogen sulfide (H₂S) in contact with a catalyst comprising a metal oxide of a metal selected from the group consisting of vanadium (V), niobium (Nb), molybdenum (Mo), chromium (Cr), rhenium (Re), tungsten (W), manganese (Mn), titanium (Ti), zirconium (Zr) and tantalum (Ta) and mixtures thereof supported on titania. Preferably, the metal oxide is supported as a monolayer. The supported metal oxide catalyst is preferably based on an oxide of vanadium (V) supported on titania. The gaseous mixture is contacted with the catalyst at a temperature of at least about 225° C. for a time sufficient to convert at least a portion of the CO and/or CO₂ and H₂S to mercaptans, primarily methanethiol. The gaseous stream containing the mercaptans is thereafter contacted with the SAPO catalyst. Hydrocarbons then are recovered as a product separate from the residual gas stream.

### EXAMPLE

To facilitate a more complete understanding of the invention, an example is provided below. The scope of the invention, however, is not to be limited to any specific embodiment disclosed in this Example, which is for purposes of illustration only.

In the following example, a SAPO catalyst (SAPO-34) was examined for its ability to convert methyl mercaptan (methanethiol) selectively to hydrocarbons generally using the following equipment and methods.

The mercaptan to hydrocarbon reaction was carried out in an isothermal fixed-bed integral mode reactor operating at atmospheric pressure. The reactor was kept in a vertical position and was made of ¼ inch O.D. quartz tube. Heating tape was used in conjunction with a feedback temperature controller (Omega CN 9000) to obtain the desired reactor temperature. SAPO-34 catalyst was obtained from Dyno Chem., Mold, Wales, U.K. in the form of powdered material. The SAPO catalyst powder (100 mg) was held at the middle of the reactor tube between porous quartz wool plugs. Prior to conducting the mercaptan reaction, the SAPO-34 catalyst was calcined in the reactor (in situ calcination) at a temperature of 500° C., using a 20 standard cubic centimeters per minute (seem) stream of 5% oxygen in helium.

Methanethiol (CH₃SH), at a concentration of 1000 ppm diluted in helium, was supplied by Scott Specialty Gases (mercaptan partial pressure of 0.76 torr). The reactant gas was sent to the reactor through glass tubing connected with Teflon fittings. Flow rates and concentrations were controlled by mass flow controllers (Brooks 5850 D, for helium and Omega FMA-767-V, 0-1 for the reactant). The lines were heated to 70° C. to prevent condensation. The total gas flow was maintained between 20 and 150 sccm. The outlet of the reactor was connected to a HP 5890 A gas chromatagraph in combination with flame ionization (FID), thermal conductivity (TCD) and sulfur chemiluminescence (SCD) detectors. This system was used to analyze the reaction products. The lines between the reactor outlet and the gas analyzers were heated to avoid condensation of the products. The flow rate of reaction products sent to the gas chromatograph was controlled by a needle valve (Nupro Company, SS-4BRG).

The SAPO-34 catalyst was contacted with a helium stream containing 1000 ppm of methanethiol at a flow rate of 25 sccm over a wide temperature range to examine the formation of hydrocarbons. Mercaptan conversions were measured at temperatures between 300 and 600° C. The exit concentration of the reaction products of the methanethiol reaction products over the SAPO-34 catalyst as a function of temperature is presented in Tables 1 and 2. As shown, methane was found to be a predominant product at the higher temperatures, though ethane and propane were also detected (Table 1). In these tests, a variety of sulfur products also were observed (Table 2), including hydrogen sulfide, dimethyl sulfide, sulfur dioxide and dimethyl disulfide. Carbon monoxide and carbon dioxide also appeared as reaction products; the formation of carbon monoxide and carbon dioxide decreased at elevated temperatures.

TABLE 1

| Catalyst | | | | | |
|---|---|---|---|---|---|
| Temperature | CH3SH Conversion | | | | Elemental C |
| (° C.) | Total | CH4 | C2H6 | C3H8 | CO/CO2/other |
| 300 | 32% | 0 ppm | 0 ppm | 1 ppm | 998 ppm |
| 400 | 43% | 4 ppm | 2 ppm | 0 ppm | 992 ppm |
| 500 | 62% | 32 ppm | 3 ppm | 0 ppm | 962 ppm |
| 600 | 92% | 188 ppm | 16 ppm | 1 ppm | 777 ppm |

TABLE 2

| Catalyst | | | | | | | |
|---|---|---|---|---|---|---|---|
| Temperature | CH3SH Conversion | | | | | | Elemental or |
| (° C.) | Total | H2S | COS | DMS | SO2 | DMDS | Deposited |
| 300 | 32% | 25% | 0% | 13% | 0% | 36% | 26% |
| 400 | 43% | 37% | 0% | 4% | 5% | 28% | 26% |
| 500 | 62% | 42% | 0% | 3% | 11% | 10% | 34% |
| 600 | 92% | 46% | 0% | 1% | 4% | 0% | 49% |

It will be understood that while the invention has been described in conjunction with specific embodiments thereof, the foregoing description and examples are intended to illustrate, but not limit the scope of the invention. Other aspects, advantages and modifications will be apparent to those skilled in the art to which the invention pertains, and these aspects and modifications are within the scope of the invention, which is limited only by the appended claims.

I/we claim:

1. A process for converting mercaptans to hydrocarbons comprising contacting a gas containing a mercaptan with a SAPO catalyst to convert at least a portion of the mercaptan to hydrocarbon and recovering said hydrocarbon.

2. The process of claim 1 wherein the SAPO catalyst is SAPO-34.

3. The process of claim I where the contacting is conducted at a temperature between 200° and 700° C.

4. The process of claim 3 wherein said contacting is conducted at a temperature between 300° and 550° C.

5. The process of claim 1 wherein said gas containing said mercaptan is contacted with said catalyst such that between 1 and 1000 standard cubic centimeters of mercaptan contacts a gram of SAPO catalyst per hour.

6. The process of claim 5 wherein said gas containing said mercaptan is contacted with said catalyst such that between 10 and 1000 standard cubic centimeters of mercaptan contacts a gram of SAPO catalyst per hour.

7. The process of claim 6 wherein said gas containing said mercaptan is contacted with said catalyst such that between 10 and 100 standard cubic centimeters of mercaptan contacts a gram of SAPO catalyst per hour.

8. The process of claim 1 wherein the mercaptan is CH₃SH.

9. A process for producing hydrocarbons comprising contacting a gas containing a mixture of carbon oxide and hydrogen sulfide with a catalyst comprising a supported metal oxide of a metal selected from the group consisting of vanadium (V), niobium (Nb), molybdenum (Mo), chromium (Cr), rhenium (Re), tungsten (W), manganese (Mn), titanium (Ti), zirconium (Zr) and tantalum (Ta) and mixtures thereof to convert said carbon monoxide and hydrogen sulfide to a mercaptan and then contacting said mercaptan with a SAPO catalyst to convert at least a portion of the mercaptan to hydrocarbon and recovering said hydrocarbon.

10. The process of claim 9 wherein the SAPO catalyst is SAPO-34.

11. The process of claim 9 wherein the contacting of the mercaptan and the SAPO catalyst is conducted at a temperature between 200° and 700° C.

12. The process of claim 11 wherein said contacting is conducted at a temperature between 300° and 550° C.

13. The process of claim 9 wherein said gas containing said mercaptan is contacted with said catalyst such that between 1 and 1000 standard cubic centimeters of mercaptan contacts a gram of SAPO catalyst per hour.

14. The process of claim 13 wherein said gas containing said mercaptan is contacted with said catalyst such that between 10 and 1000 standard cubic centimeters of mercaptan contacts a gram of SAPO catalyst per hour.

15. The process of claim 13 wherein said gas containing said mercaptan is contacted with said catalyst such that between 10 and 100 standard cubic centimeters of mercaptan contacts a gram of SAPO catalyst per hour.

16. The process of claim 9 wherein the mercaptan is CH₃SH.
